(19) United States
(12) Patent Application Publication
(10) Pub. No.: US 2010/0204802 A1
(43) Pub. Date: Aug. 12, 2010

(54) MEDICAL DEVICE

(76) Inventors: Darren ... Wilson, York (GB); Robert Morgan, York (GB)

Correspondence Address: DIANA HOUSTON;SMITH & NEPHEW, INC., 1450 BROOKS ROAD, MEMPHIS, TN, 38116, US

(21) Appl. No.: 12/441,632

(22) PCT Filed: Sep. 21, 2007
(86) PCT No.: PCT/GB07/03587

§ 371 Date: Dec. 23, 2009

(30) Foreign Application Priority Data
Sep. 21, 2006 (GB) 0618612.6

(51) Int. Cl. A61F 2/28

(52) U.S. Cl. 623/23.6

(57) ABSTRACT

The present invention provides an implant (10) comprising: an antimicrobial means (11); an activation means (4) for activating the antimicrobial means (11); and a power source (4) for powering the activation means. The present invention also provides a system comprising such an implant interfaced with a separate control means.

The present invention relates to medical devices, for example implants. In particular, the present invention relates to implants that comprise sensors and communication means. Such implants may be termed “smart implants”.

Orthopedic devices such as hip and knee implants are often prone to post-surgical infection leading to septic loosening of the implant. However, it has been hypothesized that the current incidence of low-virulent infections is underestimated because of problems of differential diagnosis. Infections associated with prosthetic joints occur less frequently than aseptic failures, but represent the most devastating complication with high morbidity and substantial cost. In addition to protracted hospitalisation, patients risk complications associated with additional surgery and antimicrobial treatment, as well the possibility of renewed disability.

Due to the absence of well-designed prospective, randomised, controlled studies with a sufficient follow-up period, diagnosis and treatment of prosthetic joint infections is mainly based on tradition, personal experience and liability aspects, and therefore differs substantially between institutions and countries. In addition, different specialists involved in the management of this complication, such as orthopedic surgeons, infectious disease physicians, and microbiologists, have different approaches.

Depending on the organism involved, infections can be either acute (symptoms appear relatively soon after material insertion) or chronic (may take months for symptoms to appear). Table 1 summarises the classification of prosthetic joint infection according to the time of symptom onset after implantation. Leading clinical signs of early infections are persisting local pain, erythema (redness in skin), oedema, wound healing disturbance, large hematoma and fever. Persisting or increasing joint pain and early loosening are the hallmarks of a delayed infection, but clinical signs of infection may be absent. Therefore, such infections are often difficult to distinguish from aseptic failure. Late infections present either with a sudden onset of systemic symptoms (in about 30%) or as a sub acute infection following unrecognised bacteraemia (in about 70%). The most frequent primary (distant) foci of implant-associated infections are skin, respiratory, dental and urinary tract infections (Zimmerli W, Ochsner P E. Management of infection associated with prosthetic joints. Infection 2003; 31:99-108; and Kaandorp C J, Dinant H J, van de Laar M A, Moens H J, Prins A P, Dijkmans B A. Incidence and sources of native and prosthetic joint infection: a community based prospective survey. Ann Rheum Dis 1997; 56:470-5).

The incidence of prosthetic joint infection is higher after a revision arthroplasty which may be due to either the long operation time, scar formation, or recrudescence of unrecognised infection present at the initial surgery. In certain cases where antibiotic treatment won't be effective, it may mean removing the implant outright, and cleaning the wound before replacing it, which is costly, both in terms of expenses, time and the patients' condition. The procedure involves a surgical incision, drainage of the pus, hardware removal and debridement of all devitalised tissue in conjunction with the long term pharmacological treatment.

Revision surgery may be associated with loss of bone stock, protracted immobilisation or rehabilitation, and peri-operative complications, especially in patients with significant co-morbidities. Moreover, treatment of an infected prosthetic joint usually exceeds the conservative estimate of $ 50000 per episode (Hebert C K, Williams R E, Levy R S, Barrack R L. Cost of treating an infected total knee replacement. Clin Orthop 1996; 140-5. 4; and Sculco T P. The economic impact of infected total joint arthroplasty. Instr Course Lect 1993; 42:349-51). This amounts to more than $ 1 billion annually.

The microorganisms begin to produce a biofilm once they attach and grow on the implant surface. Biofilms are not simply collections of individual bacteria. Instead, they are complex cooperative communities of microorganisms that contain one or more species embedded within an extracellular exopolysaccharide (EPS) matrix. It is a highly hydrated matrix of polysaccharide and protein displaying discrete temporal and spatial organization, and possessing environmental sensing mechanisms whose adaptive responses operate at the population level. Biofilms may vary widely in thickness, limited more by nutrient transport than by surface roughness. For example, aerobic Pseudomonas aeruginosa biofilms can grow to 30-40 μm in depth as monocultures, but these biofilms can increase in depth to 130 μm when the culture is amended with anaerobic bacteria (J. W. Costerton, Z. Lewandowski, D. E. Caldwell, D. R. Korber, H. M. Lappin-Scott, “Microbial biofilms,” Annu. Rev. Microbiol. 49 (1995):711-745). Biofilm bacteria can become a permanent feature of an infected device, meaning there may be no means of removing it or killing the host to eradicate the biofilm (J. W. Costerton, Philip S: Stewart, E. P. Greenberg, “Bacterial Biofilms: A Common Cause of Persistent Infections,” Science 284(21 May 1999):1318-1322). As a result, sessile biofilm bacterial communities are regarded as an irreversible infection, nearly impervious to host defense mechanisms (antibodies, phagocytes) and are difficult to detect because the extracellular sulphated 20-kD acidic polysaccharide (K. Karamanos, A. Syrokou, H. S. Panagiotopoulou, E. D. Anastassiou, G. Dimitracopoulos, “The major 20-kD polysaccharide of Staphylococcus epidermidis extracellular slime and its antibodies as powerful agents for detecting antibodies in blood serum and differentiating among slime-positive and -negative S. epidermidis and other staphylococci species,” Arch. Biochem. Biophys. 342(15 Jun. 1997):389-395.) slime matrix acts as a physical and chemical barrier to protect the bacteria from attack. It has been estimated that biofilms cause over 80% of infections. Antibiotic therapy typically reverses the symptoms caused by planktonic (individual) cells released from the biofilm, but fails to kill the biofilm itself (J. W. Costerton, Philip S. Stewart, E. P. Greenberg, “Bacterial Biofilms: A Common Cause of Persistent Infections,” Science 284(21 May 1999):1318-1322). It is estimated that bacteria within biofilms are effectively from 20-1000 times (M. J. Elder, F. Stapleton, E. Evans, J. K. Dart, “Biofilm-related infections in opthalmology,” Eye 9 (1995):102-109) to 500-5000 times (M. R. Brown, D. G. Allison, P. Gilbert, “Resistance of bacterial biofilm to antibiotics: A growth-rate related effect?” J. Antimicrob. Chemother. 20(1988):777-783) less sensitive to antibiotics than planktonic microorganisms.

Biofilms can be composed of either gram-positive or gram-negative bacteria, and species most frequently isolated from medical devices include gram-positive Enterococcus faecalis and Staphylococcus aureus, and the gram-negative Escherichia coli, Klebsiella pneumoniae and Pseudemonas aeruginosa. The bacteria can originate from patients' own skin, from the hands of healthcare workers, or from other external sources in the environment. Not only do biofilms propagate quickly but they are very difficult to control using standard methods such as antimicrobial agents. This difficulty is due to a number of factors such as restricted penetration, decreased growth rate, protection from the environment, nutrient acquisition, phenotypic variation, and intercellular communication.

TABLE 1
Classification of prosthetic joint infections according to onset
of symptoms after implantation (reproduced from Andrej Trampuza,
Werner Zimmerlib, Prosthetic joint infections: update in diagnosis
and treatment SWISS MED WKLY 2005; 135: 243-251).

| Classification | Characteristic |
|---|---|
| Early infection (<3 months) | predominantly acquired during |
| | implant surgery or the following 2 |
| | to 4 days by highly virulent |
| | organisms (e.g., Staphylococcus |
| | aureus or gram-negative bacilli) |
| Delayed or low-grade infection | predominantly acquired during |
| (3-24 months) | implant surgery and caused by |
| | less virulent organisms (e.g., |
| | coagulase-negative staphylococci |
| | or Propionibacterium acnes) |
| Late infection (>24 months) | predominantly caused by |
| | haematogenous seeding from |
| | remote infections |

The clinical methods for diagnosing orthopedic implant infection include (a) patient testimony, (b) imaging, (c) erythrocyte sedimentation rate (ESR), whole blood-cell count and C-reactive protein (CRP) levels in blood test samples, (d) synovial fluid cell count and (e) histological analysis of tissue biopsies, which are discussed in turn below.

### (a) Patient Testimony

Clinically, patients may note increasing pain at both rest and with activity accompanied by redness, swelling, and tenderness in the vicinity of the implant. Scoring methods have been used to quantify patient testimony. In particular, the McGill Pain Questionnaire (MPQ) measures a patient's subjective pain experience by using three major psychological dimensions of pain: sensory-discriminative, affective-motivational, and evaluative-cognitive (Melzack R. The McGill Pain Questionnaire: Major properties and scoring methods. Pain 1975; 1: 277-299). Clearly, such testimony is unreliable and prone to error.

### (b) Imaging

Examination of serial radiographs after implantation may be helpful, but are neither sensitive nor specific to diagnose infection (Tigges S, Stiles R G, Roberson J R. Appearance of septic hip prostheses on plain radiographs. AJR Am J Roentgenol 1994; 163:377-80). A rapid development of a continuous radiolucent line of greater than 2 mm or severe focal osteolysis within the first year is often associated with infection. Computed tomography (CT) and magnetic resonance imaging (MRI) are alternative imaging techniques. The main disadvantages of CT and MRI are imaging interferences in the vicinity of metal implants. Positron emission tomography (PET) needs further evaluation for implant imaging.

Gamma scintigraphy with Technetium-99m (^{99m}Tc) has been used to study implant infection, however, it has been reported to have a low specificity (Corstens F H, van der Meer J W. Nuclear medicine's role in infection and inflammation. Lancet 1999; 354:765-70; and Smith S L, Wastie M L, Forster I. Radionuclide bone scintigraphy in the detection of significant complications after total knee joint replacement. Clin Radiol 2001; 56:221-4). In addition, increased bone remodelling around the prosthesis is normally present during the first postoperative year and aseptic loosening cannot be differentiated from infection.

### (c) Blood Sampling

The erythrocyte sedimentation rate, the C-reactive protein serum level, and the white blood-cell count are routinely used to diagnose periprosthetic infection (Di Cesare P E, Chang E, Preston C F, Liu C J. Serum interleukin-6 as a marker of periprosthetic infection following total hip and knee arthroplasty. J Bone Joint Surg Am. 2005 September; 87(9):1921-7). Blood leukocyte count and differential are not sufficiently discriminative to predict the presence or absence of infection (Steckelberg J M, Osmon D R. Prosthetic Joint Infection. In: Bisno A L and Waldvogel F A eds. 3rd. Washington, D.C.: Am Soc Microbiol 2000:173-209). After surgery, C-reactive protein (CRP) is elevated and returns to normal within weeks. Therefore, repetitive measurements are more informative than a single value in the postoperative period.

### (d) Synovial Fluid Cell Count

Synovial fluid leukocyte count and differential is a test for differentiating prosthetic joint-associated infection from aseptic failure. A microdialysis probe can be used to withdraw a tiny sample of extracellular fluid at the site of infection. Analysis of the sample can detect the presence and amount of a variety of chemical markers such as cytokines that may indicate early signs of responses to implant infection. The limitations of this technique are that it is a relatively evasive procedure and it does not have an ideal sensitivity and specificity.

### (e) Histopatholoqical Studies

Histopathological examination of periprosthetic tissue biopsies is an evasive technique for assessing implant infection. In general, It demonstrates a sensitivity of >80% and a specificity of >90% (Trampuz A, Steckelberg J M, Osmon D R, Cockerill F R, Hanssen A D, Patel R. Advances in the laboratory diagnosis of prosthetic joint infection. Rev Med Microbiol 2003; 14:1-14). However, the degree of infiltration with inflammatory cells may vary considerably between specimens from the same patient, even within individual tissue sections. A major limitation of histopathological examination is that it does not identify the causative organism, an essential element in selection of appropriate antimicrobial therapy. In addition, interpretation of tissue histopathology from patients with underlying inflammatory joint disorders may be difficult.

In summary, no single routinely used clinical or laboratory test has been shown to achieve ideal sensitivity, specificity, and accuracy for the diagnosis of prosthetic joint infection. The major drawbacks of many of the conventional monitoring techniques are that the identities of the microbes are not accessible with these methods, and the infection is usually well developed at this point in the diagnosis. Therefore a combination of laboratory, histopathology, microbiology and imaging studies is usually required. Ideally, the infection is diagnosed (or excluded) before surgery, which enables starting antimicrobial treatment preoperatively and allows planning of the most appropriate surgical management. Despite the variety of tests available, it may also be difficult to distinguish aseptic loosening from an infected THR using conventional monitoring techniques.

According to a first aspect of the present invention, there is provided an implant comprising: a sensor; a communication means for communicating the output of the sensor; and a power source for providing power to the sensor and the communication means.

According to embodiments of the present invention, the implant further comprises a processor for processing the output of the sensor. In such embodiments, the communication means may communicate the output of the processor. The power source provides power for the processor.

According to an embodiment of the present invention, the implant further comprises a memory storage device. The memory storage device may store the output of the sensor. The memory storage device may store the output of the processor. The power source provides power for the memory storage device.

The implant may comprise one sensor. The implant may comprise a plurality of sensors. The or each sensor may be embedded in the implant.

The or each sensor may detect physical phenomena/parameters. The or each sensor may detect chemical species. The or each sensor may detect biological species.

The sensor may be a temperature sensor. The temperature sensor may measure the rise in local tissue temperature associated with inflammation.

The sensor may be a pressure sensor. The pressure sensor may measure local changes in vasodilation.

The sensor may be a load sensor. The load sensor may measure the increase in pressure exerted by the soft inflamed tissue surrounding the implant.

The sensor may be a resistor sensor. The resistor sensor may measure the change in electrical conductivity associated with oedema.

The sensor may be an electrical potential sensor. The electrical potential sensor may measure an induced bioelectric effect.

The sensor may be an oxygen sensor. The oxygen sensor may measure the growth of aerobic bacteria or bacterial infection concomitant with low oxygen tension.

The sensor may be a pH sensor. The pH sensor may monitor activity of fermenting bacteria (i.e. the drop in pH associated with biofilm infections).

The communication means may be a wireless communication means. The wireless communication means may be Zigbee. The wireless communication means may be Bluetooth. The wireless communication means may be Radio Frequency (RF). The wireless communication means may communicate the sensor output to an external reader device. The external reader device may have a memory storage device. The external reader device may be a computer.

The power source may be a battery. The power source may be an energy scavenging device. The power source may be a motion powered piezoelectric generator and associated charge storage device. The power source may be a motion powered electromagnetic generator and associated charge storage device. The power source may comprise inductively coupled systems. The power source may comprise Radio Frequency (RF) electromagnetic fields.

According to embodiments of the invention, a charge storage device may be charged with sufficient energy (for example through inductive/RF coupling or internal energy scavenging) to perform a single measurement and process and communicate the result.

The implant of the first aspect of the present invention may enable continuous monitoring of infectious agents through monitoring infection-related markers with time. Readings may be taken at home or in clinic.

The implant of the first aspect of the present invention has the advantage that it allows early detection of infection compared to conventional methods and devices. This has the associated advantage that a clinician can initiate systemic antibiotic treatment in a more timely fashion to treat the infection and prevent complications such as septic loosening. Accordingly, patient treatment is improved and optimised, with reduction/elimination of pain and suffering. Financial burden on healthcare is reduced.

According to a second aspect of the present invention, there is provided an implant comprising an antimicrobial means; an activation means for activating the antimicrobial means; a power source for powering the activation means.

In this application, an antimicrobial means is a means for disrupting, neutralising and/or eliminating microbes, including bacteria. Antimicrobial means also include means for disrupting, neutralising or eliminating a biofilm comprising microbes. Such antimicrobial means are termed disrupters, and they break up, degrade or erode biofilms.

The implant may comprise one antimicrobial means. The implant may comprise a plurality of antimicrobial means.

The disrupter may comprise physical means.

The disrupter may comprise a mechanical means. The disrupter may be a device that generates shock waves. The disrupter may be a sonication device. The disrupter may be a hydrostatic pressure device. The disrupter may comprise fluid flow.

The disrupter may comprise an electrical means. The disrupter may be an electrolysis device. The disrupter may be a voltage generator. The disrupter may be an electromagnetic generator.

The power source may be a battery. The power source may be an energy scavenging device. The power source may be a motion powered piezoelectric generator and associated charge storage device. The power source may be a motion powered electromagnetic generator and associated charge storage device. The power source may comprise inductively coupled systems. The power source may comprise Radio Frequency (RF) electromagnetic fields.

The activation means may comprise a communication means. The communication means may be a wireless communication means. The wireless communication means may be Zigbee. The wireless communication means may be Bluetooth. The wireless communication means may be Radio Frequency (RF). The wireless communication means may be interfaced with a computer.

The antimicrobial means may comprise chemical species. The antimicrobial means may comprise chemical species that disrupt, neutralise or eliminate quorum sensing signals. The antimicrobial means may comprise peroxides. The antimicrobial means may comprise O₂/O₃. The antimicrobial means may comprise iodine species. The antimicrobial means may comprise triclosan. The antimicrobial means may comprise chlorhexadene. The antimicrobial means may comprise antibiotics.

The antimicrobial means may comprise biological species. The antimicrobial means may comprise antibodies.

In those embodiments in which the antimicrobial means comprises a chemical or biological species, the implant further comprises: a storage medium for storing the chemical or biological species, the storage medium have a release mechanism for releasing the chemical or biological species, wherein the release mechanism is activated by the activation means.

The storage medium may be a reservoir embedded in the implant.

The release mechanism may be a valve.

The power source may be a battery. The power source may be an energy scavenging device. The power source may be a motion powered piezoelectric generator and associated charge storage device. The power source may be a motion powered electromagnetic generator and associated charge storage device. The power source may comprise inductively coupled systems. The power source may comprise Radio Frequency (RF) electromagnetic fields.

The control means may comprise a communication means. The communication means may be a wireless communication means. The wireless communication means may be Zigbee. The wireless communication means may be Bluetooth. The wireless communication means may be Radio Frequency (RF). The wireless communication means may be interfaced with a computer.

The implant of the second aspect of the present invention has the advantage that it allows a clinician to treat infection at the source of the infection and prevent complications such as septic loosening, without the need for systemic antibiotic treatment. If necessary, the clinician can also initiate systemic antibiotic treatment in conjunction with activation of the implant in order to treat the infection. Adcordingly, patient treatment is improved and optimised, with reduction/elimination of pain and suffering. Financial burden on healthcare is reduced.

According to a third aspect of the present invention, there is provided an implant comprising: a sensor; a communication means for communicating the output of the sensor; an antimicrobial means; an activation means for activating the antimicrobial means; and a power source for providing power to the sensor, the communication means and the activation means.

In this application, an antimicrobial means is a means for disrupting, neutralising and/or eliminating microbes, including bacteria. Antimicrobial means also include means for disrupting, neutralising or eliminating a biofilm comprising microbes. Such antimicrobial means are termed disrupters, and they break up, degrade or erode biofilms.

According to embodiments of the third aspect of the present invention, the implant further comprises a processor for processing the output of the sensor. In such embodiments, the communication means may communicate the output of the processor. The power source provides power for the processor.

According to embodiments of the third aspect of the present invention, the implant further comprises a memory storage device. The memory storage device may store the output of the sensor. The memory storage device may store the output of the processor. The power source provides power for the memory storage device.

The implant may comprise one sensor. The implant may comprise a plurality of sensors. The or each sensor may be embedded in the implant.

The or each sensor may detect physical phenomena/parameters. The or each sensor may detect chemical species. The or each sensor may detect biological species.

The sensor may be a temperature sensor. The temperature sensor may measure the rise in local tissue temperature associated with inflammation.

The sensor may be a pressure sensor. The pressure sensor may measure local changes in vasodilation.

The sensor may be a load sensor. The load sensor may measure the increase in pressure exerted by the soft inflamed tissue surrounding the implant.

The sensor may be a resistor sensor. The resistor sensor may measure the change in electrical conductivity associated with oedema.

The sensor may be an electrical potential sensor. The electrical potential sensor may measure an induced bioelectric effect.

The sensor may be an oxygen sensor. The oxygen sensor may measure the growth of aerobic bacteria or bacterial infection concomitant with low oxygen tension.

The sensor may be a pH sensor. The pH sensor may monitor activity of fermenting bacteria (i.e. the drop in pH associated with biofilm infections).

The communication means may be a wireless communication means. The wireless communication means may be Zigbee. The wireless communication means may be Bluetooth. The wireless communication means may be Radio Frequency (RF). The wireless communication means may communicate the sensor output to an external reader device. The external reader device may have a memory storage device. The external reader device may be a computer.

The implant of the third aspect may comprise one antimicrobial means. The implant may comprise a plurality of antimicrobial means.

The disrupter may comprise physical means.

The disrupter may comprise a mechanical means. The disrupter may be a device that generates shock waves. The disrupter may be a sonication device. The disrupter may be an abrasive device. The disrupter may comprise fluid flow.

The disrupter may comprise an electrical means. The disrupter may be an electrolysis device. The disrupter may be a voltage generator. The disrupter may be an electromagnetic generator.

The power source may be a battery. The power source may be an energy scavenging device. The power source may be a motion powered piezoelectric generator and associated charge storage device. The power source may be a motion powered electromagnetic generator and associated charge storage device. The power source may comprise inductively coupled systems. The power source may comprise Radio Frequency (RF) electromagnetic fields.

The activation means may comprise a communication means. The communication means may be a wireless communication means. The wireless communication means may be Zigbee. The wireless communication means may be Bluetooth. The wireless communication means may be Radio Frequency (RF). The wireless communication means may be interfaced with a computer.

The antimicrobial means may comprise chemical species. The antimicrobial means may comprise chemical species that disrupt, neutralise or eliminate quorum sensing signals. The antimicrobial means may comprise peroxides. The antimicrobial means may comprise O₂/O₃. The antimicrobial means may comprise iodine species. The antimicrobial means may comprise triclosan. The antimicrobial means may comprise chlorhexadene. The antimicrobial means may comprise antibiotics.

The antimicrobial means may comprise biological species. The antimicrobial means may comprise antibodies.

In those embodiments in which the antimicrobial means comprises a chemical or biological species, the implant further comprises: a storage medium for storing the chemical or biological species, the storage medium have a release mechanism for releasing the chemical or biological species, wherein the release mechanism is activated by the activation means.

The storage medium may be a reservoir embedded in the implant.

The release mechanism may be a valve.

The power source may be a battery. The power source may be an energy scavenging device. The power source may be a motion powered piezoelectric generator and associated charge storage device. The power source may be a motion powered electromagnetic generator and associated charge storage device. The power source may comprise inductively coupled systems. The power source may comprise Radio Frequency (RF) electromagnetic fields.

According to embodiments of the invention, a charge storage device may be charged with sufficient energy (for example through inductive/RF coupling or internal energy scavenging) to perform a single measurement and process and communicate the result.

The implant of the third aspect of the present invention may enable continuous monitoring of infectious agents through monitoring infection-related markers with time. Readings may be taken at home or in clinic.

The implant comprises antimicrobial means that can be activated to disrupt, neutralise or eliminate microbes, including bacteria. The antimicrobial means can disrupt, neutralise or eliminate a biofilm comprising microbes.

The implant may respond automatically to the sensed data and activate the antimicrobial means.

The implant may automatically communicate the sensed data to an external control means. The external control means may be a computer. The external control means may automatically communicate with the activation means to activate the antimicrobial means. The external control means may provide a user, for example a clinician, with the sensed data. The clinician may then activate the antimicrobial means.

The external control means may operate a surgical treatment algorithm. The surgical treatment algorithm may be as hereinafter described.

The implant may automatically communicate the sensed data to an internal processor in the implant. The internal processor may automatically communicate with the activation means to activate the antimicrobial means.

The internal processor may operate a surgical treatment algorithm. The surgical treatment algorithm may be as hereinafter described.

The implant of the third aspect of the present invention has the advantage that it allows early detection of infection compared to conventional methods and devices. It also has the advantage that it allows a clinician to treat infection at the source of the infection by activation of the antimicrobial means and prevent complications such as septic loosening, without the need for systemic antibiotic treatment. If necessary, the clinician can also initiate systemic antibiotic treatment in conjunction with activation of the implant in order to treat the infection. Accordingly, patient treatment is improved and optimised, with reduction/elimination of pain and suffering. Financial burden on healthcare is reduced.

According to a fourth aspect of the present invention, there is provided a system comprising an implant according to the first aspect of the present invention interfaced with a separate control means. The control means may be a computer.

According to a fifth aspect of the present invention, there is provided a system comprising an implant according to the second aspect of the present invention interfaced with a separate control means. The control means may be a computer.

According to a sixth aspect of the present invention, there is provided a system comprising an implant according to the third aspect of the present invention interfaced with a separate control means. The control means may be a computer.

The implant of any of the first, second, third, fourth, fifth, or sixth aspects of the present invention may be any type of suitable implant. Examples of implants in accordance with this invention comprise, but are not limited to, the following: (a) reconstructive devices, the tibial, femoral, or patellar components used in total knee replacement, the femoral or acetabular components used in total hip implants, the scapular or humeral components in shoulder replacement, the tibia and talus in ankle replacement, and between the vertebral bodies in the lumbar and cervical spine disk replacements, the humerus, ulna and radius in elbow replacement, and metacarpals and carpals in finger joints; and (b) trauma devices (nail, plate, bone screw, cannulated screw, pin, rod, staple and cable). The invention also includes dental and craniomaxillofacial implant applications.

Embodiments of the invention have the advantage that the implant or system allows for information to be gathered and processed yielding useful clinical data with respect to implant infection. They also allow the clinician to intervene in a more timely fashion using a technology that is specifically designed to inhibit biofilm production on the surface of an implant and improve the efficacy of antibiotic therapy. The invention enables the reduction of infection rates following surgery and significantly reduces health care costs while improving the quality of life for patients.

Reference will now be made, by way of example, to the accompanying drawings, in which:

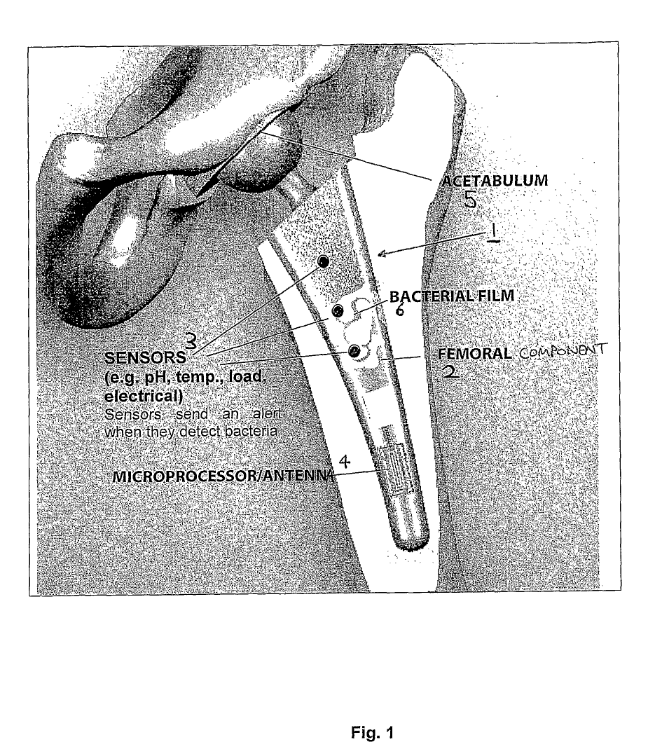

FIG. 1 is a schematic representation of an orthopedic hip implant in accordance with an embodiment of the present invention;

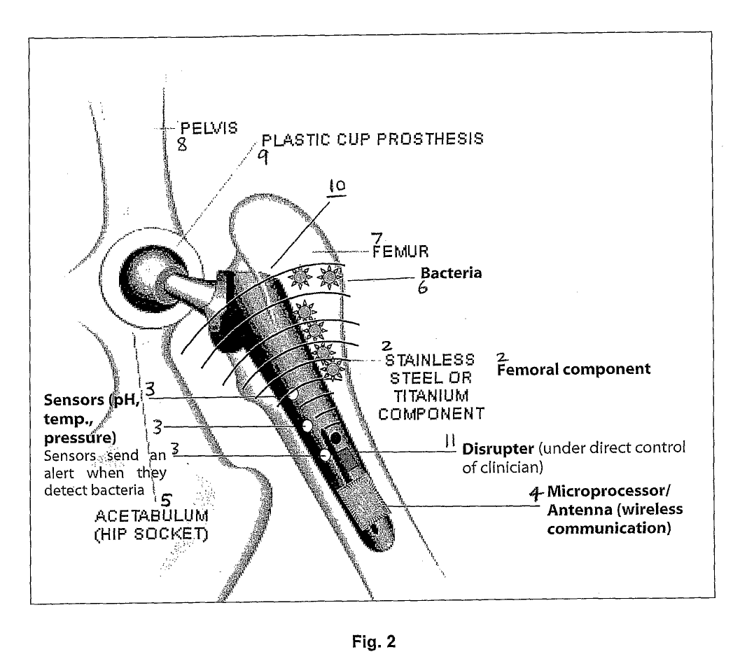

FIG. 2 is a schematic representation of an orthopedic hip implant in accordance with an embodiment of the present invention;

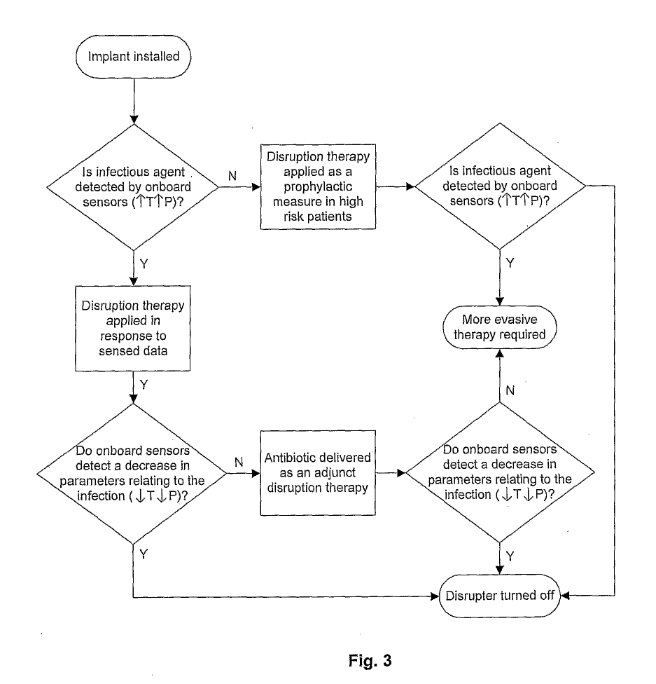

FIG. 3 is a flow chart specifying a decision making process of an orthopedic device in accordance with an embodiment of the present invention; and

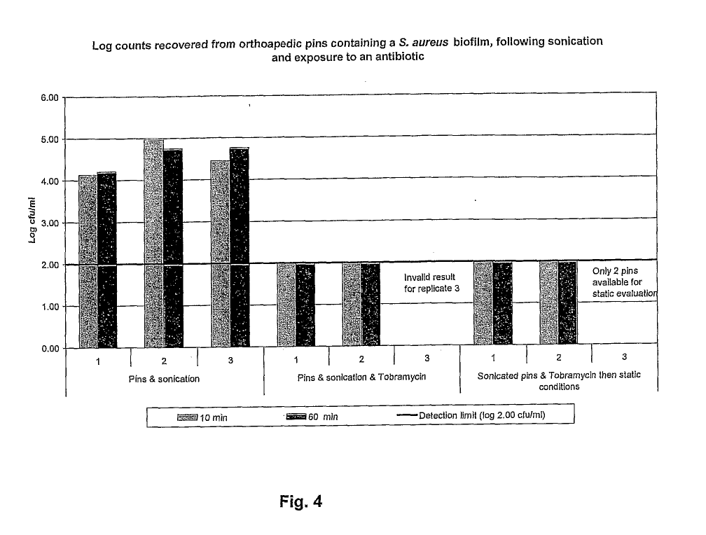

FIG. 4 shows log counts recovered from orthopedic pins containing S. aureus biofilm, following sonication and exposure to an antibiotic.

FIG. 1 shows an orthopedic hip implant (1) in accordance with an embodiment of the present invention. The implant (1) comprises a femoral component (2), sensors (3), and a microprocessor/antenna (4). The sensors (3) may be pH, temperature, load, or electrical sensors, for example. As shown in FIG. 1, the femoral component (2) has a bacterial film (6) on its outer surface. The sensors (3) send an alert via the microprocessor/antenna (4) when they detect bacteria (6). The particular implant shown is a hip implant (1) for implantation in a femur (7) and acetabulum (5).

FIG. 2 shows an orthopedic hip implant (10) in accordance with an embodiment of the present invention. The implant (10) comprises a femoral component (2), sensors (3), a microprocessor/antenna (4), and an antimicrobial means/disrupter (11). The sensors (3) may be pH, temperature, load, or electrical sensors, for example. As shown in FIG. 2, the femoral component (2) has bacteria (6) on its outer surface. The sensors (3) send an alert via the microprocessor/antenna (4) when they detect bacteria (6).

The antimicrobial means/disrupter (11) may be any of those disclosed herein. The antimicrobial means/disrupter (11) may be under the direct control of a clinician.

The particular implant shown in FIG. 2 is a hip implant (10) for implantation in a femur (7) and acetabulum (5). In the embodiment shown in FIG. 2, the acetabulum (5) accommodates a plastic cup prosthesis (9).

The implants of FIG. 1 or 2 can have femoral components (2) made of stainless steel or titanium, for example.

The microprocessor/antenna (4) enables wireless communication. The microprocessor/antenna (4) may comprise a power source (not shown). The power source may power the sensors (3) and/or the antimicrobial means/disrupter (11).

According to some embodiments of the present invention, an external power source may be used, as described herein.

### Intelligent Implant/System

An implant in accordance with embodiments of the present invention is a smart orthopedic total hip replacement (THR) which is instrumented to carry out the following operations in a closed loop system: (a) sense and store data relating to implant infection (e.g. temperature, pressure, oxygen consumption and pH), (b) apply intelligence to decide whether or not action is required by the patient/clinician in response to the sensed data using a surgical treatment algorithm developed specifically for processing information relating to joint infection, (c) activate a telemetry link to alert the clinician of an impending infection enabling him/her to apply a disruption therapy to eradicate the biofilm and improve the delivery of antibiotic therapy.

Implants according to embodiments of the present invention consist of sensors and associated electronic components located in machined cavities on the outer surface of a hip stem and the femoral head. A hermetically sealed housing is adapted for implantation in the body of a patient. Incorporation of sensors and other electronic components within an implantable medical device such as a THR alters its primary function from a passive (load-supporting device) to a smart “intelligent” system with the ability to record and monitor aseptic loosening. The sensor inputs, through addition of telemetric capabilities, triggers a therapeutic response from within the implant that affects its environment. The application of telemetry enables implantable devices to sense changes in their environment and either predict or confirm the appropriateness of any particular function of the implant in situ. The data may be stored and relayed to a central monitoring station. A simple alert signal may be activated to trigger a further response from either the implantable or externally worn disruption module in order to directly affect the environment of the implant, such as infection.

### Sensors

An implant/system for early monitoring of implant infection in accordance with embodiments of the present invention is schematically illustrated in FIGS. 1 and 2. FIG. 1 illustrates an implant comprising sensors. FIG. 2 illustrates an implant comprising sensors and an antimicrobial means in the form of a disrupter. The implants provide continuous, non-destructive data pertaining to the biofilm process and can function in an aqueous environment, not require sample removal, minimise signal from organisms or contaminants in the bulk phase and provide real time data.

The implants consist of an array of sensors strategically positioned at or near the implant which can simultaneously measure parameters relating to implant infection. The ability to record multiple parameters simultaneously improves the decision making process regarding the management of infectious agents. Preferred sensors are predictive, having the ability to detect the presence, counts of and/or identification of bacterial cells/endotoxins in bodily fluid/biofilm on an implant's surface. The sensors alert the patient/clinician of an impending infection using an accurate, non-invasive, in-situ monitoring solution which occurs without any obvious clinical signs of infection. The sensing elements enable long term monitoring of patients in either the hospital or work environment.

The sensors assess the degree of tissue inflammation surrounding an orthopedic implant (e.g. hip, knee, ankle or shoulder) which occurs in response to a deep wound infection. Infection results in inflammation and accompanying heat generation and therefore changes in local tissue temperature. As a result, temperature sensors provide early conformation indications of the presence of deep infections associated with septic loosening since infection results in a rise in local tissue temperature. Inflammation also results in an increase in pressure in the local tissue, vasodilation and increased vascular permeability. A pressure sensor may measure the increase in arterial blood pressure which occurs in the tissue surrounding an infected implant. A load sensor may measure the increase in pressure exerted by the soft inflamed tissue surrounding the implant. As tissue fibre bundles come under uniaxial tension, this force may be detected by a nearby load sensor probe. Sensors responsive to fluid flow, concentration of analytes, pH and other variables may also be used to improve the decision making algorithm. A pH sensor may be used for the determination of the presence of pathological conditions associated with abnormal pH levels, particularly those associated with the activity of fermenting bacteria. An oxygen sensor may measure the growth of aerobic bacteria or bacterial infection concomitant with low oxygen tension (L. E. Bermudez, M. Petrofsky, and J. Goodman, “Exposure to low oxygen tension and increased osmolarity enhance the ability of Myobacterium avium to enter intertestinal epithelial (HT-29) cells,” Infect. Immun., vol. 65, no. 9, pp. 3768-3773, September 1997). Biochemical sensors may be used to monitor the raised inflammatory markers, which occurs in response to the infection. When bacterial cells adhere to the surface of the implant, the phenotype of the bacteria changes and polysaccharides are released which have specific markers which could be detected by sensors.

Other indicators of a deep infection include the measurement of time-dependent changes in the electrical potential across an implant surface. Biofilm activity can alter the interfacial chemistry and thereby change the potential of the biosystem. Under a defined set of conditions, a change in potential measured can be used to indicate microbial activity and may correlate with the presence of biomass. A deep infection can cause tissue oedema, and so the increase in water content is likely to cause a change in the electrical conductivity across the surface of the implant which may be measured using a resistor sensor.

With this knowledge obtained from the sensor array, the clinician may determine the best course of treatment of the patient such as (a) the prescription of antibiotics, (b) the activation of the implant antimicrobial means to remove the biofilm which could also be applied as a preventative or prophylactic measure, (c) simultaneous application of antimicrobials and inhibition therapy or (d) a more suitable time for re-operation.

Suitable sensors include those that can continuously sense their environment and collect data. This may need to continue for years after surgery. The sensors may measure parameters relating to implant infection (e.g. temperature, load, pressure, pH and oxygen) and transmit a reading when queried by a receiver in a remote intelligence monitor. A temperature sensor is sensitive enough to detect local changes in temperature associated with deep infections. A load sensor is capable of measuring the increase in tension in the tissue bundle fibres which occurs during tissue inflammation. A pressure sensor is capable of measuring local changes in blood pressure associated with inflammation and deep infections. A pH sensor is used for the determination of the presence of pathological conditions associated with abnormal pH levels, particularly those associated with the activity of fermenting bacteria. An oxygen sensor will measure the growth of aerobic bacteria or bacterial infection concomitant with low oxygen tension. The sensors are sensitive enough to warn of a spike in vital functions that might lead to implant infection. By heeding the instant information and making the appropriate response, such as activating the disruption therapy, patients can avert a health crisis. The sensors can be self-powered using piezoelectric elements or externally powered by either electromagnetic induction, radio frequency (RF) induction or batteries.

The sensors are positioned at or near the surface of the implant and are arranged in such a way that they can detect infectious agents across the entire surface of the implant.

### Antimicrobial Means/Disruption Apparatus

Implants in accordance with embodiments of the present invention use Intelligent microelectrical mechanical systems (MEMS) devices to provide either mechanical (e.g. ultrasound, sonication, shock waves), electrical (e.g. voltage, electrolysis) or biological/chemical (e.g. enzyme treatment) stimulation to inhibit/disrupt biofilm formation, disperse adherent bacteria from the surface, and disrupt their multicellular structure. The activation of these intelligent devices may be under direct control of the clinician and can be either implantable or externally worn devices. These disruption therapies may be used in conjunction with prophylactic antibiotics improving its efficacy/mode of action.

In embodiments of the present invention, the clinician may pulse the infected implant with ultrasound delivered using either an internal piezoelectric sensor attached to or embedded in the implant surface or by using a portable hand held system positioned on the skin of the infected tissue. Ultrasound is acoustic (sound) energy in the form of waves having a frequency above the human hearing range. The highest frequency that the human ear can detect is approximately 20,000 Hz. This is where the sonic range ends, and where the ultrasonic range begins. The top end of the frequency range is limited only by the ability to generate the signals—frequencies in the gigahertz range (upwards of 1 billion cycles per second) have been used. A single exposure of focused ultrasound energy is called a “sonication.” The term “sonochemistry” is defined as chemistry enhanced by intense sound waves. The main event in sonochemistry is the creation, growth and collapse of a bubble that is formed in the liquid. It is well known that in a sonication bath, with a power of 0.3 W/cm², water is converted to hydrogen peroxide. The sonochemical yield and rate is dependent upon parameters such as (frequency, power, gas under which the sonication takes place, pressure of the gas, temperature.)

Pulsed ultrasound perturbs bacterial cell membranes by cavitation, which stimulates the active and/or passive uptake of antibiotics. This method enables the specific targeting and eradication of bacterial cells in and near biofilms.

In other embodiments of the present invention, the MEMS device may deliver an electric field across the surface of the implant in order to enhance the efficacy of charged biocides and antibiotics in eradicating biofilm bacteria. The radio frequency electromagnetic field acts directly on the polar parts of the molecules forming the biofilm matrix, reducing metabolic activity and growth rate of the bacteria maximising antibiotic efficacy.

In other embodiments of the present invention, the implant is equipped with a microprocessor modulated delivery system which controls the release of biological/chemical manipulations on demand based on sensed data preventing biofilm formation. An algorithm may be used to electronically control the elution rate of a therapeutic agent which is stored in gated reservoir located in the implant. The biological manipulation could act specifically on cell density-dependent or “quorum-sensing signals,” which is one form of communication between bacteria. Quorum-sensing signals are important in coordinating multicellular behaviour in bacteria and regulate a number of physiological processes. Disruption of this signalling pathway would prevent a biofilm from developing on the surface of the implant.

The active agent of the delivery system may be a germicide or antiseptic agent such as nitric oxide, silver nitride or a peroxide compound which is known to be effective against a wide variety of bacteria. A surfactant such as ethylenediaminetetraacetic acid (EDTA) may be used which targets the breakdown of polysaccharides, a major constituent of the biofilm.

The active therapeutic module pre-implanted with the sensor may be further adapted such that it activates the release of an antibiotic that blocks the formation of biofilms or dismantles them such that the intrinsic resistance of biofilms to antibiotics is eliminated and the infection can be better eradicated.

### Closed Feedback Loop

Implants and systems in accordance with the present invention are capable of diagnosing and treating nascent bacterial infections, as well as providing feedback to the patient and physician via a telemetry link. A flow chart highlighting the surgical treatment algorithm for a prosthetic joint infection is shown in FIG. 2. The invention provides a feedback loop and means for modifying the implant after placement in a patient in response to measurements made by the array of onboard sensors. The parameters of interest are processed by an algorithm in such a way that they can be interpreted by either the patient or the clinician. Feedback from the sensor(s) alerts the clinician to whether any action is required in a more timely fashion than current diagnostic methods. The clinician can then decide whether or not to act upon the sensed data by activating the implant antimicrobial means (for example on-board disruption therapies) and simultaneously administer an antibiotic when more evasive treatment is required to eradicate the infection. The treatment algorithm also accounts for high risk patients where the disruption therapy is applied as a prophylactic.

### Power Sources

Septic loosening can occur after the index of surgery or to up to 15 years to 20 years thereafter. It is therefore important that the system functions longitudinally in time. Power management strategies may include implanted power sources, e.g. batteries or may make use of energy scavenging devices, such as motion powered piezoelectric or electromagnetic generators and associated charge storage devices. Other forms of power supply may include inductively coupled systems or Radio Frequency (RF) electromagnetic fields. It may also be possible to charge a storage device with sufficient energy (either through inductive/RF coupling or internal energy scavenging) to perform a single shot measurement and to subsequently process and communicate the result.

### Data Management

Implants and systems in accordance with the invention may be used to connect a patient to a remote data storage system, such as the internet or a computer accessible through devices such as PDA, phone system devices that the physician or nurse can monitor or use to interact remotely with the implant.

### Communication

Implants and systems according to the present invention have the ability to transmit stored information by wireless communication using available technologies such as Zigbee, Bluetooth or Radio Frequency (RF). ZigBee is a published specification set of high level communication protocols designed for wireless personal area networks (WPANs). Bluetooth is a technical industry standard that facilitates short range communication between wireless devices. RF is a wireless communication technology using electromagnetic waves to transmit and receive data using a signal above approximately 0.1 MHz in frequency.

### Telemetrized Orthopedic Implant

According to an embodiment of the present invention, there is provided a telemetrized orthopedic implant. The implant continuously measures a set of values relating to implant infection and then transmit them from the implant to a reader device without disturbing its primary function which is to support load. The implant may be manufactured with a recess up to 0.5 mm thick to protect the sensors and conductor wires from abrasive damage during the surgical insertion process. A sensor may be fixed to the surface of the metal cavity using a range of high stiffness adhesives including epoxy resins, polyurethanes, uv curable adhesives, and medical grade cyanoacrylates. These fixation methods do not adversely affect the performance of the sensor.

There are a number of ways to encapsulate the electronics. If a battery or other potentially hazardous device is included in the electronics system a titanium case may be utilised. Alternatively, if the components are biologically benign, then a simple potting material e.g., a biocompatible potting material, may be utilised. Biocompatible potting materials include materials such as polyurethane or silicone which provide a hermetic seal. Since the electronic components are sealed hermetically from the patient tissues and fluids, long term function of the device is achieved. At the same time, leakage of non-biocompatible or toxic materials is eliminated. The potting material is an electrically insulative, moisture resistant material, supplied in either a liquid or putty-like form and is used as a protective coating on sensitive areas of electrical and electronic equipment. The sensors and conductors may be covered in a potting material with suitable mechanical characteristics required to survive the implantation process and restore the mechanical envelope. The implant also includes electronic components forming an instrumentation circuit with the sensors. Therefore, the remaining electrical components such as the PCB are also housed in machined cavities and covered in a biocompatible potting material in order to prevent contact with body fluids and moisture.

In those embodiments of the invention comprising antimicrobial means comprising biological/chemical species for disruption therapy administered on demand based on the outcome of the sensed data, the auxiliary reservoirs used to contain the species are inserted into a hollow central portion of the femoral component of a total hip replacement. These specialised designed femoral components are prepared using novel additive manufacturing processes in preference to conventional machining and forging techniques.

Implants in accordance with embodiments of the present invention provide real-time, objective and accurate data on the detection of infectious agents that are responsible for biofilm formation without disturbing the primary function of the implant. Once the infectious agent is detected by the sensors, the instrumentation allows the clinician to activate a therapeutic modality which is designed specifically to prevent a biofilm from developing on the surface of the implant. This prevents the patient from undergoing a potentially painful and costly revision surgery to replace the infected implant. The in-vivo, low cost, solution can be used in the doctor's office or at the patient's home.

Examples of implants in accordance with the present invention comprise, but are not limited to, the following: (a) reconstructive devices (the tibial, femoral, or patellar components used in total knee replacement), the femoral or acetabular components used in total hip implants, the scapular or humeral components in shoulder replacement, the tibia and talus in ankle replacement, and between the vertebral bodies in the lumbar and cervical spine disk replacements, the humerus, ulna and radius in elbow replacement, and metacarpals and carpals in finger joints) and (b) trauma devices (nail, plate, bone screw, cannulated screw, pin, rod, staple and cable). The instrumentation could also be extended to dental and craniomaxillofacial implant applications.

According to some embodiments of the present invention there is provided at least one sensor as hereinbefore described housed within a self-contained unit that patrols the implant described above from a nearby position once it is deployed by the surgeon, perhaps using a minimally invasive surgical technique.

The at least one sensor may be in the form of a self-contained chip. The chip may be based on RFID technology.

The self-contained chip may have a miniaturized, wireless implantable sensor and an external electronics module (external to the patient). The external electronics module wirelessly communicates with the sensors to deliver vital patient data.

The wireless sensor may be powered by Radio Frequency (RF) energy transmitted from an external electronics module and may transmit real-time data. The external electronics module may comprise a reader system.

The chip may comprise a hermetically sealed circuit encapsulated in materials such as boro-silicate glass and silicone, for example. The chip may be surrounded by a PTFE-coated nickel-titanium wire.

The chip may be 15 to 25 mm long. The chip may be 17 to 23 mm long. The chip may be 19 to 21 mm long. The chip may be around 20 mm long.

The chip may be 3 to 10 mm wide. The chip may be 3 to 7 mm wide: The chip may be 4 to 6 mm wide. The chip may be around 5 mm wide.

The chip may be 3 to 10 mm deep/thick. The chip may be 3 to 7 mm deep. The chip may be 4 to 6 mm deep. The chip may be around 5 mm deep.

The following scenarios are within the scope of the present invention.

A patient receives a wireless instrumented joint reconstruction product. The electromechanical system within the implant may be used to monitor patient recovery using one or more sensors, and make a decision as to whether any anti-microbial therapy is required during the rehabilitation period. Alternatively, the therapeutic module may be permanently activated as a preventative measure.

The technology associated with the instrumentation procedure may also be adapted to monitor other implant-related infections related to the cardiovascular system, urinary tract, and surgical wounds.

The instrumented device may also be used as a research tool to allow the clinician to perform in vivo monitoring and diagnostics of orthopedic and other implants for the general patient in order to understand the mechanism of septic loosening.

FIG. 4 shows log counts recovered from orthopedic pins containing a S. aureus biofilm, following sonication and exposure to an antibiotic. Sterile stainless steel orthopedic pins (10 mm length×6 mm diameter) were pre-incubated with 1 ml heat inactivated horse serum for 30 minutes at 37° C. with agitation. The pins were then inoculated with a culture of S. aureus (clinically relevant bacterium) prepared in tryptone soya broth to contain approximately 10⁷ cfu/ml and incubated at 37° C. for 72 hours with agitation. Pins were then removed from the culture, washed to remove any planktonic bacterial cells and then enumerated at 10 and 60 minutes after:

sonication in diluent alone;

sonication in diluent with 4 μg/ml Tobramycin; and

initial sonication in diluent for 10 minutes then addition of 4 μg/ml Tobramycin and left in static conditions

It was pre-determined that sonication of such pins in diluent at 60 kHz for 10 minutes was sufficient to remove the non-planktonic bacteria present on the pin surface. Extending the time to 60 minutes allowed the effect of continued sonication either with or without antibiotic to be studied.

The results show that sonication in the presence of Tobramycin (@ 4 μg/ml) reduces cell numbers to the detection limit within 10 minutes. Continued sonication was not required for Tobramycin to reduce cell numbers to the detection limit within 10 minutes, once the cells were removed from the pin by an initial sonication period of 10 minutes.

This demonstrates that sonication and exposure to an antibiotic is efficacious at disrupting and eliminating an in vitro bacterial biofilm. Hence, implants and systems according to embodiments of the present invention utilising sonication as an antimicrobial means in conjunction with an antibiotic are efficacious at disrupting and eliminating a bacterial biofilm. Sonication alone, using optimised frequencies and exposure times, is also efficacious at disrupting and eliminating a bacterial biofilm.

1. An implant comprising:
an antimicrobial means;
an activation means for activating the antimicrobial means; and a power source for powering the activation means.

2. An implant according to claim 1, wherein the implant comprises a plurality of antimicrobial means.

3. An implant according to claim 1, wherein at least one of the antimicrobial means comprises a disrupter for disrupting microbes.

4. An implant according to claim 1, wherein at least one of the antimicrobial means comprises a mechanical means.

5. An implant according to claim 1, wherein at least one of the antimicrobial means comprises a chemical species.

6. An implant according to claim 1, wherein at least one of the antimicrobial means comprises a biological species.

7. An implant according to claim 3, wherein the antimicrobial means is selected from the group consisting of a shock wave generating device, a sonication device, a hydrostatic pressure device, an electrical means, an electrolysis device, a voltage generator, and an electromagnetic generator.

8. A device according to claim 5, wherein the antimicrobial means is selected from the group consisting of peroxides, oxygen, ozone, iodine species, triclosan, chlorhexadene and antibiotics.

9. A device according to claim 6, wherein the antimicrobial means comprises antibodies.

10. An implant according to claim 1, further comprising:
a sensor; and
a communication means for communicating the output of the sensor, wherein the power source provides power to the sensor and the communication means.

11. An implant according to claim 10, further comprising a processor for processing the output of the sensor, wherein the communication means communicates the output of the processor and the power source provides power for the processor.

12. An implant according to claim 10, further comprising a memory storage device, wherein the memory storage device stores the output of the sensor and/or the output of the processor, and wherein the power source provides power for the memory storage device.

13. An implant according to claim 10, wherein the implant comprises a plurality of sensors.

14. An implant according to claim 10, wherein at least one of the sensors detects physical phenomena.

15. An implant according to claim 10, wherein at least one of the sensors detects chemical species.

16. An implant according to claim 10, wherein at least one of the sensors detects biological species.

17. An implant according to claim 10, wherein the sensor is selected from the group consisting of a temperature sensor, a pressure sensor, a load sensor, a resistor sensor, an electrical potential sensor, an oxygen sensor and a pH sensor.

18. An implant according to claim 1, wherein the activation means comprises a communication means.

19. An implant according to claim 10, wherein the communication means is a wireless communication means.

20. An implant according to claim 19, wherein, in use, the wireless communication means communicates the sensor output to an external reader device.

21. An implant according to claim 1, wherein the power source is selected from the group consisting of a battery, an energy scavenging device, a motion powered piezoelectric generator and associated charge storage device, a motion powered electromagnetic generator and associated charge storage device, an inductively coupled system and radio frequency coupling.

22. An implant according to claim 1, wherein the implant is selected from the group consisting of reconstructive implants, trauma implants, dental implants and craniomaxillofacial implants.

23. An implant according to claim 1, wherein the implant comprises a chip.

24. (canceled)

25. A system comprising an implant according to claim 1 interfaced with a separate control means.

26. A system according to claim 25, wherein the control means is a computer.

27. (canceled)
